(19) United States
(12) Patent Application Publication
(10) Pub. No.: US 2019/0345553 A1
(43) Pub. Date: Nov. 14, 2019

(54) ESTIMATION OF THE AGE OF DRIED BIOLOGICAL FLUID STAINS UTILIZING RNA DEGRADATION AND qPCR TECHNIQUES AND KITS AND METHODS OF USE RELATED THERETO

(71) Applicant: The Board of Regents for Oklahoma State University, Stillwater, OK (US)

(72) Inventors: Robert W. Allen, Tulsa, OK (US); Jun Fu, Tulsa, OK (US)

(21) Appl. No.: 16/279,407

(22) Filed: Feb. 19, 2019

Related U.S. Application Data
(60) Provisional application No. 62/710,397, filed on Feb. 16, 2018.

(51) Int. Cl. C12Q 1/6876; C12Q 1/686

(52) U.S. Cl. CPC C12Q 1/6876; C12Q 1/686

(57) ABSTRACT

Methods and kits for estimating the age of a biological fluid sample.

### CROSS REFERENCE TO RELATED APPLICATIONS

This application claims benefit under 35 USC § 119(e) of provisional application U.S. Ser. No. 62/710,397, filed Feb. 16, 2018. The entire contents of the above-referenced application are expressly incorporated herein by reference.

### STATEMENT REGARDING FEDERALLY FUNDED RESEARCH OR DEVELOPMENT

This invention was made with U.S. Government support under NIJ Grant No. 2014-DN-BX-K025 awarded by the Department of Justice. The Government has certain rights in the invention.

### TECHNICAL FIELD

The presently disclosed and claimed inventive concept(s) relate to method(s) and kit(s) for quantifying and/or estimating the amount of time a biological fluid sample (either liquid or dried) has been deposited at a scene and/or the estimated age of the biological fluid sample. More specifically, the presently disclosed and claimed inventive concept(s) relate to embodiments of a method for determining the estimated age of a biological fluid sample (either liquid or dried) via measuring the level and/or amount of degradation of mRNA transcripts and/or portions thereof over time amplified via quantitative polymerase chain reaction (qPCR), as well as kits and methods of use related thereto.

### BACKGROUND

It is common practice for forensic laboratories and forensic experts to quantitate the amount of human genomic DNA recovered from evidentiary biological fluid samples, as well as an identification of the gender of the DNA-donor. Moreover, forensic experts are often called into court to testify as to when a particular biological fluid sample was deposited, for instance, by way of example only, deposited at a crime scene. The time of deposit of a biological fluid sample remains a critical component of DNA experts' testimonies—testimonies that can facilitate the conviction or exoneration of an individual(s). Current method(s) for quantifying and/or estimating the age of a biological fluid sample utilize two transcripts—one transcript in which the amount of the transcript is expected to change over time, and a second transcript in which the amount of the transcript is expected to remain stable. This dual monitoring of transcripts allows the quantity of the changing transcript to be normalized by comparison to the quantity of the stable transcript among biological and technical replicates. However, one primary problem with the current methodology is that stochastic effects can occur during the reverse transcription reaction and again during qPCR cycles that may dramatically affect the estimates of complementary DNA (cDNA) produced, thereby negatively affecting the reproducibility of quantitation estimates for cDNA copies of the RNA transcripts present in a particular biological fluid sample. For instance, if one of the transcripts is more abundant than the other, this problem may be magnified due to a more abundant transcript not being affected as significantly as a less abundant transcript.

Accordingly, there is a need for improved method(s) and kit(s) that, at least: (1) accurately establish the time a biological fluid sample was deposited at a scene; (2) accurately establish the duration of time that has elapsed since the biological fluid sample was deposited at a scene; (3) allow for the quantification of nucleic acids present in a particular biological fluid sample (either liquid or dried); and/or (4) utilize a single transcript, for instance, by way of example only, a single mRNA transcript to establish the above. It is to such and methods and kits that the presently disclosed and claimed inventive concept(s) is directed.

### BRIEF DESCRIPTION OF THE DRAWINGS

FIG. 1 is a non-limiting visual representation showing the locations of qPCR primers relative to coding and non-coding regions of LGALS2, CLC, S100A12, and B2M.

FIG. 2A is a non-limiting graphical plot showing degradation kinetics of LGALS2 transcripts in bloodstains stored for up to one year.

FIG. 2B is a non-limiting graphical plot showing degradation kinetics of CLC transcripts in bloodstains stored for up to one year.

FIG. 2C is a non-limiting graphical plot showing degradation kinetics of S100A12 transcripts in bloodstains stored for up to one year.

FIG. 2D is a non-limiting graphical plot showing degradation kinetics of B2M transcripts in bloodstains stored for up to one year.

FIG. 3 depicts a non-limiting graphical plot establishing the disappearance of two mRNA transcripts LGALS2 and serine peptidase inhibitor, Kazal type 2 (SPINK2) as detected by both a TaqMan methodology and the presently disclosed and/or claimed inventive concept(s), including, without limitation, the 5′-3′ method, over a storage period of fifteen (15) months.

FIG. 4 depicts a graphical plot which illustrates the results of pooled data obtained from conducting the presently disclosed and/or claimed inventive concept(s), including, without limitation, the 5′-3′ method, performed with RNA transcripts, such as, by way of example, LGALS2 mRNA transcripts, extracted from dried blood stains prepared and assayed on different dates over a period of 35 months.

FIG. 5 depicts a graphical plot of data collected for LGALS2 and B2M mRNA transcripts utilizing the presently disclosed and/or claimed inventive concept(s), including, without limitation, 5′-3′ method, over a period of twelve (12) weeks, as well as regression lines related thereto.

FIG. 6 demonstrates the effect of temperature and humidity on the degradation rate of the S100A12 transcript over a period of 16 weeks.

FIG. 7 shows ΔCq for S100A12 transcript as quantified in bloodstains stored under varying conditions, such as at 37° C. and 75% relative humidity, 4° C. and 10% relative humidity conditions after 30 days.

### DETAILED DESCRIPTION

Before explaining at least one embodiment of the inventive concept(s) in detail by way of exemplary drawings, experimentation, results, and laboratory procedures, it is to be understood that the inventive concept(s) is not limited in its application to the details of construction and the arrangement of the components set forth in the following description or illustrated in the drawings, experimentation and/or results. The inventive concept(s) is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

Unless otherwise defined herein, scientific and technical terms used in connection with the presently disclosed and claimed inventive concept(s) shall have the meanings that are commonly understood by those of ordinary skill in the art. Further, unless otherwise required by context, singular terms shall include pluralities and plural terms shall include the singular. The foregoing techniques and procedures are generally performed according to conventional methods well known in the art and as described in various general and more specific references that are cited and discussed throughout the present specification. The nomenclatures utilized in connection with, and the laboratory procedures and techniques of, analytical chemistry, synthetic organic chemistry, and medicinal and pharmaceutical chemistry described herein are those well-known and commonly used in the art.

All patents, published patent applications, and non-patent publications mentioned in the specification are indicative of the level of skill of those skilled in the art to which this presently disclosed and claimed inventive concept(s) pertains. All patents, published patent applications, and non-patent publications referenced in any portion of this application are herein expressly incorporated by reference in their entirety to the same extent as if each individual patent or publication was specifically and individually indicated to be incorporated by reference.

All of the devices, kits, and/or methods disclosed and claimed herein can be made and executed without undue experimentation in light of the present disclosure. While the compositions and methods of this presently disclosed and claimed inventive concept(s) have been described in terms of preferred embodiments, it will be apparent to those of skill in the art that variations may be applied to the compositions and/or methods and in the steps or in the sequence of steps of the method described herein without departing from the concept, spirit and scope of the presently disclosed and claimed inventive concept(s). All such similar substitutes and modifications apparent to those skilled in the art are deemed to be within the spirit, scope and concept of the inventive concept(s) as defined by the appended claims.

As utilized in accordance with the present disclosure, the following terms, unless otherwise indicated, shall be understood to have the following meanings:

The use of the word “a” or “an” when used in conjunction with the term “comprising” in the claims and/or the specification may mean “one,” but it is also consistent with the meaning of “one or more,” “at least one,” and “one or more than one.” The singular forms “a,” “an,” and “the” include plural referents unless the context clearly indicates otherwise. Thus, for example, reference to “a compound” may refer to 1 or more, 2 or more, 3 or more, 4 or more or greater numbers of compounds. The term “plurality” refers to “two or more.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects. For example but not by way of limitation, when the term “about” is utilized, the designated value may vary by ±20% or ±10%, or ±5%, or ±1%, or ±0.1% from the specified value, as such variations are appropriate to perform the disclosed methods and as understood by persons having ordinary skill in the art. The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y and Z” will be understood to include X alone, Y alone, and Z alone, as well as any combination of X, Y and Z. The use of ordinal number terminology (i.e., “first”, “second”, “third”, “fourth”, etc.) is solely for the purpose of differentiating between two or more items and is not meant to imply any sequence or order or importance to one item over another or any order of addition, for example.

As used in this specification and claim(s), the terms “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As used herein, the term “substantially” means that the subsequently described event or circumstance completely occurs or that the subsequently described event or circumstance occurs to a great extent or degree. For example, the term “substantially” means that the subsequently described event or circumstance occurs at least 90% of the time, or at least 95% of the time, or at least 98% of the time.

As used herein, the phrase “associated with” includes both direct association of two moieties to one another as well as indirect association of two moieties to one another. Non-limiting examples of associations include covalent binding of one moiety to another moiety either by a direct bond or through a spacer group, non-covalent binding of one moiety to another moiety either directly or by means of specific binding pair members bound to the moieties, incorporation of one moiety into another moiety such as by dissolving one moiety in another moiety or by synthesis, and coating one moiety on another moiety.

The term “biological fluid sample” as used herein will be understood to include any type of biological fluid sample that may be utilized in accordance with the presently disclosed and claimed inventive concept(s). Examples of biological samples that may be utilized include, but are not limited to, whole blood or any portion thereof (i.e., plasma or serum), saliva, sputum, cerebrospinal fluid (CSF), vaginal fluid, intestinal fluid, intraperotineal fluid, cystic fluid, sweat, interstitial fluid, tears, mucus, urine, bladder wash, semen, combinations, and the like. The volume of the biological fluid sample utilized in accordance with the presently disclosed and claimed inventive concept(s) is from about 1 to about 100 microliters. As used herein, the term “volume” as it relates to the liquid test sample utilized in accordance with the presently disclosed and claimed inventive concept(s) means from about 0.1 microliter to about 100 microliters, or from about 1 microliter to about 75 microliters, or from about 2 microliters to about 60 microliters, or less than or equal to about 50 microliters, or less than or equal to about 40 microliters. In one non-limiting embodiment of the presently disclosed and/or claimed inventive concept(s), the biological fluid sample comprises whole blood, saliva, semen, and vaginal fluid.

The term “housekeeping gene(s)” as used herein will be understood to include any and all constitutive genes (i.e., genes that are continually transcribed) that are required for the maintenance of basic and normal cellular functioning and which are typically expressed in all cells of an organism under normal and patho-physiological conditions. Housekeeping genes include, but are not limited to, genes coding for glyceraldehyde 3-phosphate dehydrogenase (GAPDH), beta-2 microglobulin (B2M), actin beta (ACTB), and/or ribosomal ribonucleic acid (rRNA) species, such as, by way of example only, 18S ribosomal ribonucleic acid (18S rRNA). In accordance with the presently disclosed and/or claimed inventive concept(s), housekeeping genes are important for the calculation of RT-PCR and/or qPCR ΔCq values. Cq may be defined as the cycle threshold or the number of PCR cycles required for a fluorescent signal to cross a predetermined threshold level for a sequence of interest (e.g., the fluorescent signal exceeds a background level). Cq levels are inversely proportional to the amount of target nucleic acid in a sample meaning that that the lower the Cq level, the greater the amount of target nucleic acid in said sample. ΔCq represents the difference in the genetic expression of two (2) genes and/or genetic sequences. In one non-limiting embodiment of the presently disclosed and/or claimed inventive concept(s), ΔCq is calculated by either (1) calculating the difference between the Cq of a particular sequence of interest and the Cq of a reference sequence or (2) measuring the difference between the abundance of the 5′ end of a single transcript and the 3′ end of the same single transcript. In one non-limiting embodiment, the reference sequence is a housekeeping gene sequence, such as, by way of example only 18S rRNA. In addition, in one non-limiting embodiment, the sequence of interest is selected from the group comprising and/or consisting of genes and/or genetic sequences encoding for galectin 2 (LGALS2), Charcot-Leyden crystal galectin (CLC), beta-2-microglobulin (B2M), and/or S100 calcium binding protein A12 (S100A12).

The term “patient” includes human and veterinary subjects. In certain embodiments, a patient is a mammal. In certain other embodiments, the patient is a human. “Mammal” for purposes of treatment refers to any animal classified as a mammal, including human, domestic and farm animals, nonhuman primates, and zoo, sports, or pet animals, such as dogs, horses, cats, cows, etc.

The term “transcript(s)” includes RNA strands (including, without limitation, mRNA) representing complementary copies to genetic information stored in strands of DNA that have undergone the process of transcription with the aid of RNA polymerase(s). In addition, the term “transcriptome” refers to the total amount of transcripts present in a particular cell.

Turning now to particular embodiments, the presently disclosed and claimed inventive concept(s) relate to method(s) and kit(s) estimating the age of a biological fluid sample, for instance, a dried or liquid biological fluid sample deposited at a current or former crime scene. More specifically, the presently disclosed and claimed inventive concept(s) relate to embodiments of a method for estimating the age of a particular biological fluid sample via quantifying the degradation of the 5′ and/or 3′ ends of a single ribonucleic acid transcript, including, without limitation, mRNA transcripts, as well as kits related thereto.

It is contemplated that virtually any reagent used in the fields of biological, chemical, or biochemical analyses and assays could be used in the devices, kits, and methods of the presently claimed and disclosed inventive concept(s). It is contemplated that these reagents may undergo physical and/or chemical changes when bound to an analyte of interest whereby the intensity, nature, frequency, or type of signal generated by the reagent-analyte complex is directly proportional or inversely proportional to the concentration of the analyte existing within the fluid sample. These reagents may contain indicator dyes, labeled nucleic acid probe molecules, metal, enzymes, polymers, antibodies, and electrochemically reactive ingredients and/or chemicals that, when reacting with an analyte(s) of interest, may exhibit change in color.

Any method of detecting and measuring the analyte in a fluid sample can be used in the devices, kits, and methods of the presently claimed and inventive concepts, including without limitation, polymerase chain reaction (PCR) techniques, such as, by way of example only, real-time polymerase chain reaction (RT-PCR) and/or quantitative polymerase chain reaction (qPCR) techniques. A variety of assays for detecting analytes are well known in the art and include, but are not limited to, chemical assays, DNA and/or RNA probe or capture assays, enzyme inhibition assays, antibody stains, latex agglutination, latex agglutination inhibition, and immunoassays, such as, radioimmunoassays.

In one non-limiting embodiment, the presently disclosed and/or claimed inventive concept(s) relate to method(s) and kit(s) that, for instance, via utilizing RNA sequencing of the transcriptome(s) within biological fluid sample(s) (either liquid or dried), identify a number of mRNA transcripts that disappear from the transcriptomes of stains of the biological fluid samples at, for instance, room temperature over specific intervals in a measured time period.

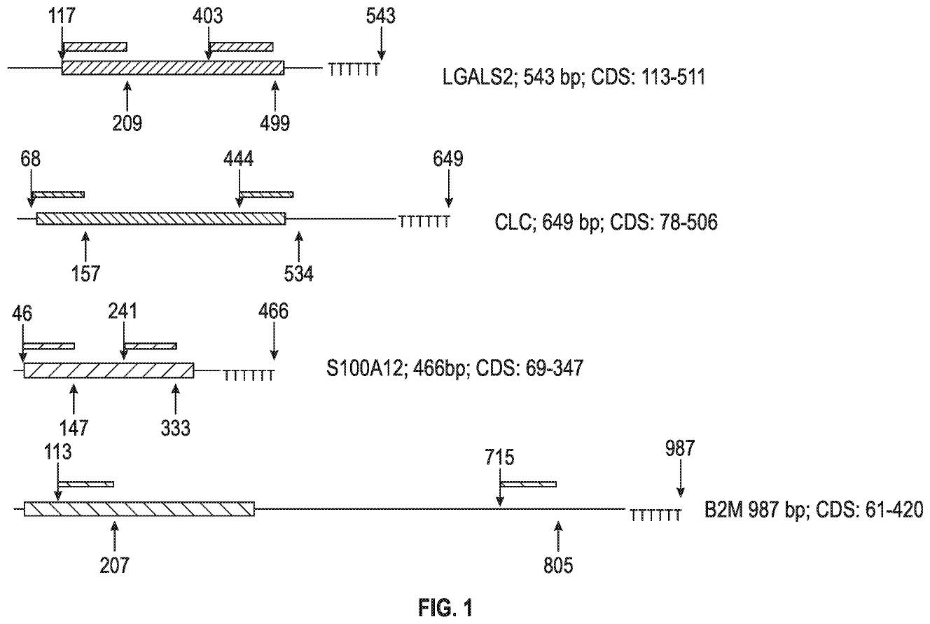

One non-limiting aspect of the presently disclosed and/or claimed inventive concept(s) relates to the RNA sequencing read depth in a number of transcripts of interest in which it is determined that the 5′ end of the transcript(s) degrade(s) more rapidly than the 3′ end of the transcript(s) and that such degradation of the 5′>3′ ends of the transcript(s) proceeds in a predictable and reproducible manner. In such method(s) and kit(s), primers are designed to detect a short stretch or portion of the 5′ end of the specific transcript of interest and a second pair of primers is designed to amplify the 3′ end of the same specific transcript, wherein each amplicon is about 100 base pairs in length. A non-limiting embodiment depicting the amplification process of various transcripts and/or sequences of interest (LGALS2, CLC, B2M, and S100A12) is shown in FIG. 1. Primer locations are shown above the sequences of the cDNAs. Heavy solid lines represent coding regions for each mRNA, whereas thin lines represent non-coding regions located at the 5′ and 3′ ends of a transcript. Also shown is non-limiting embodiment of the length of each transcript as well as the span of the coding region denoted at the starting and ending base positions. While the presently disclosed and/or claimed inventive concept(s) are described with respect to specific ribonucleic acid transcripts (such as, by way of example only, LGALS2, CLC, B2M, and/or S100A12), a person having ordinary skill in the art should readily appreciate that any number or type of transcripts may be used and the degradation characteristics of such transcripts may be utilized in accordance with the presently disclosed and/or claimed inventive concept(s) to estimate the age of a biological fluid sample. As shown in FIG. 1, primers are designed to quantify the 5′ and 3′ ends of selected mRNA transcripts (which, in one non-limiting embodiment and as depicted in FIG. 1, comprise and/or consist of LGALS2, CLC, B2M, and S100A12 transcripts) present in dried biological fluid samples/stains, for instance, by way of example only, dried blood stains. With specific reference to FIG. 1, the heavier solid lines represent coding sequences within the specific transcript while the lighter lines represent untranslated sequences at the 5′ or 3′ ends of the mRNA transcripts. This amplification and quantitation methodology may be referred to herein as the “5′-3′ method.”

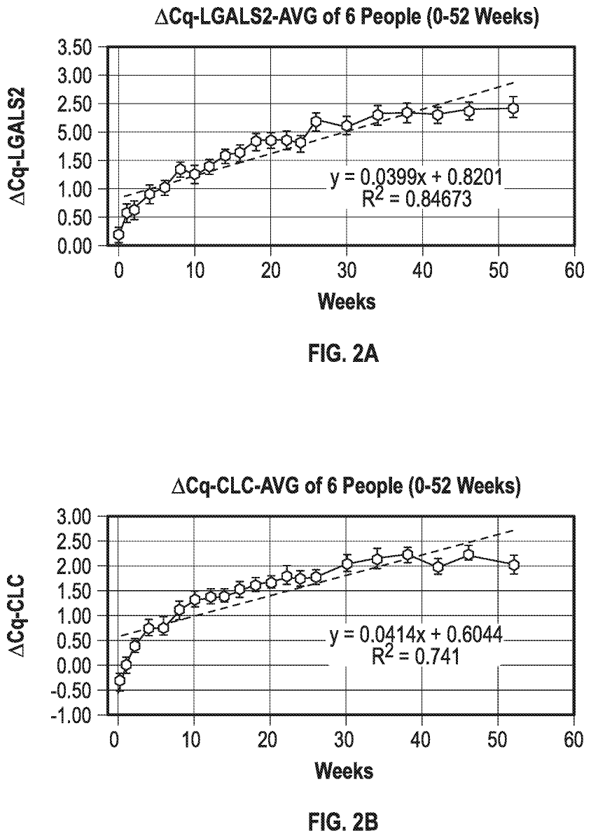

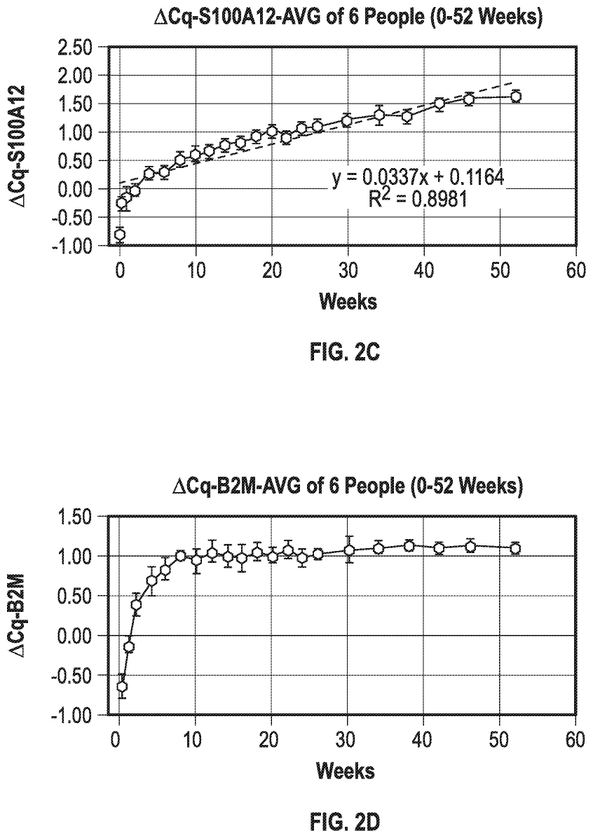

Referring now to FIG. 2, shown therein is a non-limiting graphical plot showing degradation kinetics of LGALS2, CLC, S100A12, and B2M transcripts in bloodstains stored for up to one year. Data in each figure represents average ΔCq values (+SD) from 6 unrelated blood donors (three males and three females). ΔCq represents the abundance of the 5′ and 3′ ends of a specific transcript and is measured as the Cq of the 5′ amplicon minus the Cq of the 3′ amplicon of a transcript. As shown in FIG. 2, the ΔCq values for three of four transcripts at time zero (TO) are close to zero which is to be expected for relatively intact mRNA. However, for B2M the ΔCq value at time zero was consistently a negative value. Thereafter, the ΔCq value rises rapidly up to about 8 weeks of storage and then levels off rising only slightly over the course of one year of storage. The ΔCq values for LGALS2, CLC, and B2M level off during the storage time course suggesting that either the abundance of transcript fragments from the 5′ end drop below the level of detection or that the degradation of fragments from the 3′ end of the transcript becomes equal to that of the 5′ end of the transcript, or both. The storage time at which leveling of the curve commences differs for the different transcripts. For LGALS2, the curve begins to level at about 24 weeks of storage whereas for CLC the curve begins to level at about 10 weeks of storage. The 5′ end of the B2M transcript degrades very quickly over the first 6-8 weeks of storage and then degradation kinetics become flat for the remainder of the year. These kinetics are striking when compared with the other markers whose degradation curves continue to rise over the year of storage. The leveling off of the curve does not appear to be due to a loss of the transcript fragment from the 5′ end of the molecule inasmuch as the Cq values for the 5′ amplicon average 17-20, well above the detection threshold for the qPCR instrument.

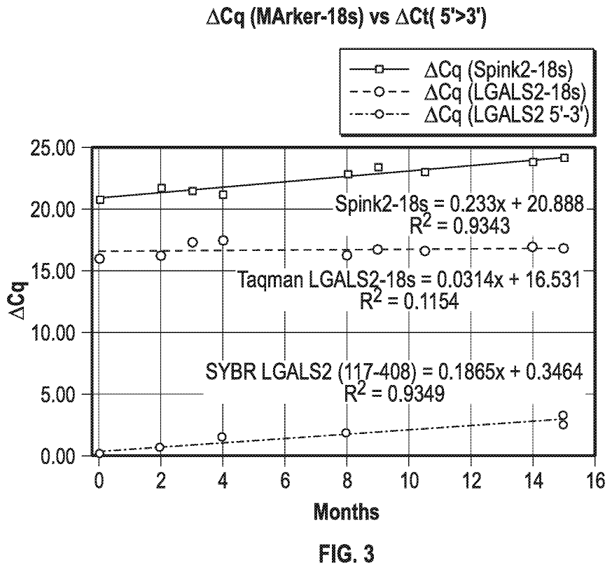

Referring now to FIG. 3, shown therein is a graphical plot establishing the disappearance of two mRNA transcripts LGALS2 and serine peptidase inhibitor, Kazal type 2 (SPINK2) as detected by both a TaqMan methodology (commonly known in the art) and the presently disclosed and/or claimed inventive concept(s), including, without limitation, the 5′-3′ method, over a storage period of fifteen (15) months. As shown in FIG. 3, the mRNA transcript LGALS2 was quantified over a storage period of fifteen (15) months: (1) by using the TaqMan methodology in which the amounts of LGALS2 and 18S rRNA were quantified to produce a ΔCq value (which, in this instance, is calculated as the difference between the Cq of LGALS2 at each time point and the Cq of 18S rRNA); or (2) by quantifying the abundance of fragments of the single LGALS2 transcript utilizing the 5′-3′ method to produce a ΔCq value (which, in this instance, is calculated as the difference between the abundance of the 5′ end of the LGALS2 transcript and the 3′ end of the LGALS2 transcript).

As can be seen in FIG. 3, the T0 ΔCq values for the TaqMan curves for LGALS2 and SPINK2 are both greater than 15, underscoring the significant difference in the abundance of LGALS2 and/or SPINK2 and 18S rRNA. In contrast, in accordance with the presently disclosed and/or claimed inventive concept(s), the 5′-3′ method produces a ΔCq at T0 that is close to zero (0), thereby demonstrating the relatively equal abundance of the 5′ and 3′ LGALS2 mRNA fragments. In addition, as shown in FIG. 3, the results from the TaqMan method for LGALS2 suggest that there is no change in the amount of LGALS2 transcript over the measured period of time. The results from the 5′-3′ method for LGALS2, conversely, suggest that: (i) the LGALS2 transcript does in fact degrade and disappear the longer the LGALS2 transcript is stored; and (ii) the kinetics of the degradation and disappearance of the 5′ end of the LGALS2 transcript are more reproducible than the points on the regression line produced using the TaqMan method. As previously stated herein, the 5′-3′ method produces more accurate data because any stochastic effects that occur during reverse transcription are mitigated, eliminated, and/or normalized due to the target of the 5′-3′ method being a single mRNA transcript rather than two (as required by the TaqMan method and previously-used methodologies in the industry).

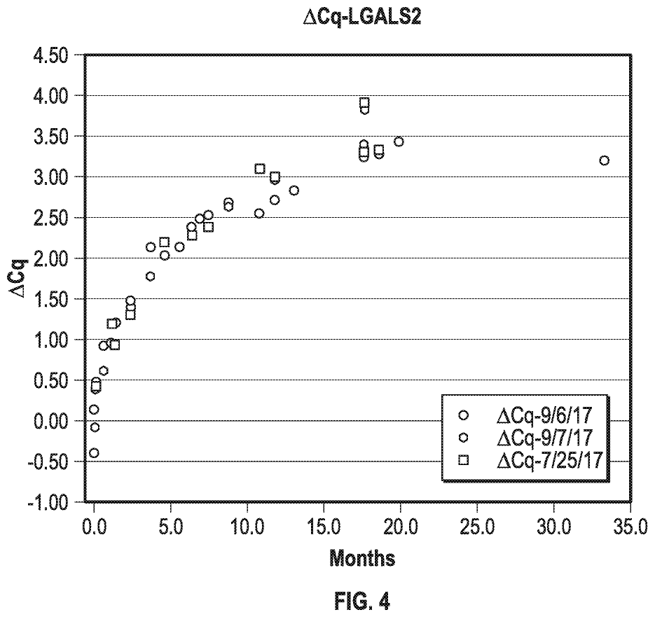

Reproducibility of the results obtained from utilizing the 5′-3′ method can additionally be measured by comparing the ΔCq values from technical replicates of a particular transcript, for instance, by way of example only, a LGALS2 transcript (i.e., qPCR experiments performed at different times for a particular mRNA transcript). FIG. 4 illustrates the results of pooled data obtained from the conducting the 5′-3′ method performed with RNA transcripts, such as, by way of example, LGALS2 mRNA transcripts, extracted from dried blood stains prepared and assayed on different dates over a period of 35 months. As shown in FIG. 4, the reproducibility of results obtained utilizing the presently disclosed and/or claimed inventive concept(s) is high.

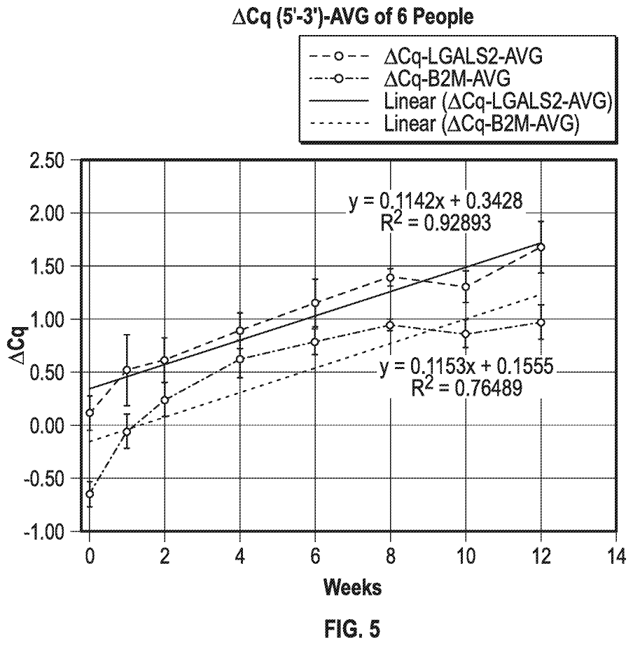

The 5′-3′ method has also been utilized to assay mRNA transcripts, including, without limitation, LGALS2 and/or B2M mRNA transcripts, in biological fluid samples, including, dried blood stains, for shorter aging periods to examine the degradation of the particular mRNA transcript over a shorter period of time. Referring now to FIG. 5, shown therein are data collected for LGALS2 and B2M mRNA transcripts utilizing the 5′-3′ method over a period of twelve (12) weeks, as well as regression lines related thereto. As can be seen in FIG. 5, the reproducibility of the results obtained utilizing the 5′-3′ method is high.

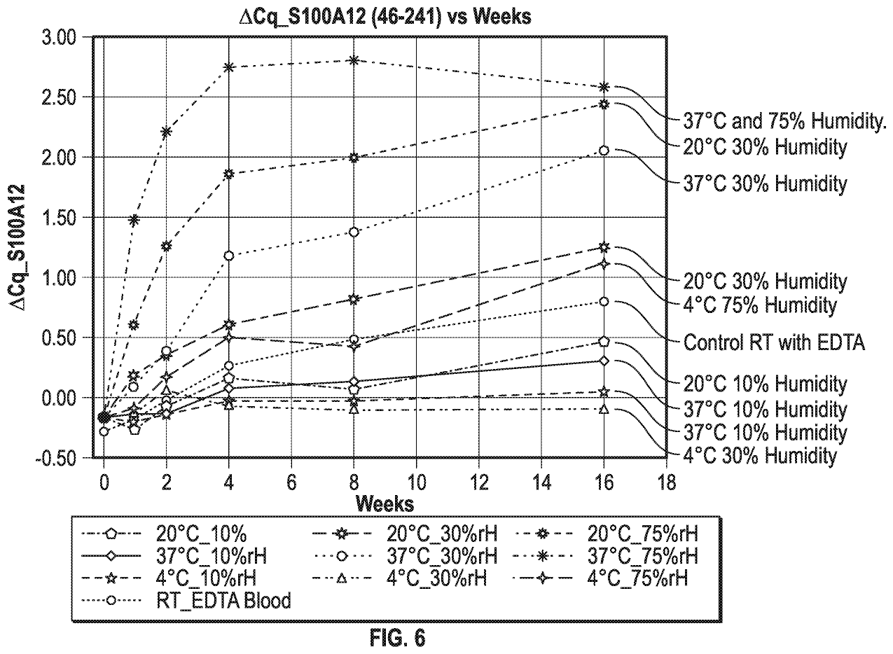

Referring now to FIG. 6, shown therein is the effect of various temperature and humidity conditions on the degradation rate (ΔCq) of a S100A12 transcript over a period of 16 weeks. As can be seen from FIG. 6, the degradation rate of the S100A12 transcript increases as the temperature and humidity are increased.

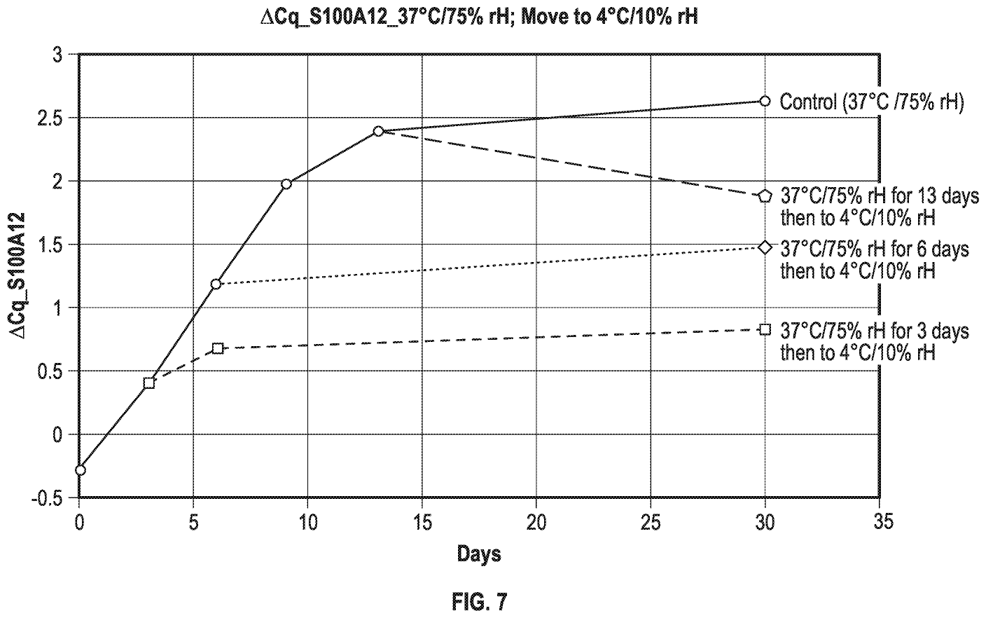

Referring now to FIG. 7, shown therein is the degradation rate (ΔCq) of a S100A12 transcript in bloodstains that were subjected to the following conditions over a 30 day-period: (1) bloodstains were created and placed at 37° C. at about 75% humidity; (2) after 3 days at the previous conditions, a portion of the stains were moved to conditions of 4° C. at about 10% humidity and were maintained at these conditions for the duration of the 30 day-period; and (3) other portions of the stains remaining in the 37° C./˜75% humidity conditions were moved to conditions of 4° C. and about 10% humidity at days 6 and 13, respectively. Accordingly, the S100A12 transcript was quantified in each of the stain populations under all conditions at 30 days. As can be seen from FIG. 7, there is direct relationship between the degradation rate of the S100A12 transcript and the temperature and humidity conditions of the stain—the higher the temperature and humidity conditions are for the stain, the higher the degradation rate of the S100A12 transcript.

Certain non-limiting embodiments of the presently disclosed and/or claimed inventive concept(s) include, but are not limited to the following:

A method for estimating the age of a biological fluid sample by detecting and quantifying the degradation of a ribonucleic acid transcript, the method comprising the steps of: designing a first pair of primers to amplify a portion of a 5′ end of a ribonucleic acid transcript present in a biological fluid sample; designing a second pair of primers to amplify a portion of a 3′ end of the ribonucleic acid transcript present in the biological fluid sample; amplifying the portion of the 5′ end of the ribonucleic acid transcript present in the biological fluid sample utilizing the first pair of primers and the portion of the 3′ end of the ribonucleic acid transcript in the biological sample utilizing the second pair of primers; quantifying the degradation of the ribonucleic acid transcript over a period of time to determine a point in time in which the ribonucleic acid transcript is present or not present in the biological fluid sample; and estimating an age of the biological sample based on the present or non-presence of the ribonucleic acid transcript in the biological fluid sample.

The method as disclosed and/or claimed herein, wherein the portion of the 5′ end of the ribonucleic acid transcript is about 100 base pairs.

The method as disclosed and/or claimed herein, wherein the portion of the 3′ end of the ribonucleic acid transcript is about 100 base pairs.

The method as disclosed and/or claimed herein, wherein the ribonucleic acid transcript comprises a messenger ribonucleic acid transcript.

The method as disclosed and/or claimed herein, wherein the messenger ribonucleic acid transcript is selected from the group consisting of for galectin 2 (LGALS2), Charcot-Leyden crystal galectin (CLC), S100 calcium binding protein A12 (S100A12), beta-2-microglobulin (B2M), and combinations thereof.

The method as disclosed and/or claimed herein, wherein the biological fluid sample is selected from the group consisting of whole blood, blood plasma, blood serum, saliva, sputum, cerebrospinal fluid (CSF), vaginal fluid, intestinal fluid, intraperotineal fluid, cystic fluid, sweat, interstitial fluid, tears, mucus, urine, bladder wash, semen, and combinations thereof.

The method as disclosed and/or claimed herein, wherein the biological fluid sample is whole blood.

The method as disclosed and/or claimed herein, wherein the biological fluid sample is a dried sample.

The method as disclosed and/or claimed herein, wherein the biological fluid sample is a liquid sample.

The method as disclosed and/or claimed herein, wherein the portion of the 5′ end of the ribonucleic acid transcript and the portion of the 3′ end of the ribonucleic acid transcript are amplified via RT-PCR or qPCR.

The method as disclosed and/or claimed herein, wherein the period of time is in a range of from about 0 days to about 365 days.

The method as disclosed and/or claimed herein, wherein the degradation of the ribonucleic acid transcript is quantified by calculating a ΔCq value for the ribonucleic acid transcript.

The method as disclosed and/or claimed herein, wherein the ΔCq value is calculated by subtracting an amount of the 5′ end of the ribonucleic acid transcript from the amount of the 3′ end of the ribonucleic acid transcript.

A kit for estimating the age of a biological fluid sample by detecting and quantifying the degradation of a ribonucleic acid transcript, comprising: a first pair of primers to amplify a portion of a 5′ end of a ribonucleic acid transcript present in a biological fluid sample; a second pair of primers to amplify a portion of a 3′ end of the ribonucleic acid transcript present in a biological fluid sample; and a printed correlation index that details degradation rates of common ribonucleic acid transcripts over a period of time.

The kit as disclosed and/or claimed herein, wherein the portion of the 5′ end of the ribonucleic acid transcript is about 100 base pairs.

The kit as disclosed and/or claimed herein, wherein the portion of the 3′ end of the ribonucleic acid transcript is about 100 base pairs.

The kit as disclosed and/or claimed herein, wherein the ribonucleic acid transcript comprises a messenger ribonucleic acid transcript.

The kit as disclosed and/or claimed herein, wherein the common ribonucleic acid transcripts are selected from the group consisting of galectin 2 (LGALS2), Charcot-Leyden crystal galectin (CLC), S100 calcium binding protein A12 (S100A12), beta-2-microglobulin (B2M), and combinations thereof.

The kit as disclosed and/or claimed herein, wherein the biological fluid sample is selected from the group consisting of whole blood, blood plasma, blood serum, saliva, sputum, cerebrospinal fluid (CSF), vaginal fluid, intestinal fluid, intraperotineal fluid, cystic fluid, sweat, interstitial fluid, tears, mucus, urine, bladder wash, semen, and combinations thereof.

The kit as disclosed and/or claimed herein, wherein the biological fluid sample is a dried sample.

The kit as disclosed and/or claimed herein, wherein the biological fluid sample is a liquid sample.

### NON-LIMITING EXAMPLES OF THE INVENTIVE CONCEPT(S)

### Example 1

In one non-limiting embodiment of the presently disclosed and/or claimed inventive concept(s), four transcripts were chosen based primarily on their relative abundance in newly prepared bloodstains (initial Cq=13-25 depending upon the particular transcript) and on the rate of their disappearance from RNA sequencing results. Transcripts chosen include: (1) LGALS2, encoding a beta-galactoside binding lectin-like protein known as galectin; (2) CLC, encoding a lysophospholipase expressed in eosinophils and basophils; (3) S100A12 encoding a calcium binding protein; and (4) B2M encoding the small protein beta-2-microglobulin associated with histocompatibility antigens. Both the presence of LGALS2 and CLC transcripts is primarily restricted to blood. The S100A12 transcript is present in blood, vaginal secretions, and saliva, and the B2M transcript is generally expressed in all body fluids. Gene transcripts and the primers designed to amplify them using qPCR are summarized in Table 1 hereinbelow. The primers utilized were specific for their intended targets as verified by searching the NCBI website using Blast analysis. Forward primers for the 5′ amplicon produced from each transcript were designed to span exon boundaries, whereas the primers directing amplification of the 3′ amplicon were not be designed to span exon boundaries. Primers were synthesized commercially (Invitrogen, Carlsbad, Calif.) and were designed to have comparable melting temperatures. A map of primer locations relative to the coding and non-coding regions of each of the transcripts is shown in FIG. 1.

TABLE 1
Gene Transcripts and Associated Primer Sequences Utilized for Amplification

| Transcript | Reference | Product | Span | Sequence (5′ [symbol] ) | | PCR Effi- | | |
|---|---|---|---|---|---|---|---|---|
| (bp) | sequence | size | [symbol] ? | (SEQ ID NO:) | | ciency (%) | Length | Start |
| LGALS2( [symbol] bp) | NM_D [symbol] | | | | | | | |
| | 5′ end | 93 | yes | CGGG [symbol] GAGG [symbol] AAGA | (1) | 101 | 21 | 117 |
| | | | | TTACAAAGCCATCA [symbol] | (2) | | 22 | 209 |
| | 3′ end | 92 | no | ATGGGCACGAGCTGACT [symbol] | (3) | 103 | 20 | 408 |
| | | | | CTTGAAAGAGGACATGTTGAACCC | (4) | | 24 | 499 |
| CLC (649 bp) | NM_ [symbol] 01828 | | | | | | | |
| | 5′ end | 90 | yes | GGAGACAACAATGTCCCTGCT | (5) | 97 | 21 | [symbol] |
| | | | | AGTGGTCGCCCTTTGATTGTC | (6) | | 21 | 157 |
| | 3′ end | 91 | no | ATGGTGCAAGTGTGGAGAGA [symbol] | (7) | 103 | 21 | [symbol] |
| | | | | AGGGATTCCTTGGCAACATGA | (8) | | 2 [symbol] | [symbol] 34 |
| S1 [symbol] 12 | NM_005621.1 | | | | | | | |
| (466 bp) | 5′ end | 102 | yes | GGGGT [symbol] AACAT [symbol] AGG [symbol] | (9) | 97 | 21 | 46 |
| | | | | TGTCAAAATGCCCCTTCCGA | (10) | | 20 | 247 |
| | 3′ end | 92 | no | TCCAAGGCCTGGATGCTAATC | (11) | 99 | 21 | 241 |
| | | | | TGTGGTAATGGGCAGCCT [symbol] C | (12) | | 20 | 333 |
| 82M (927 bp) | NM_004048.2 | | | | | | | |
| | 5′ end | 9 [symbol] | yes | TGGAG [symbol] TATCCAGCGTACT | (13) | 101 | 20 | 313 |
| | | | | CCCAGACACATAGCAATTCAGG | (14) | | 27 | 207 |
| | 3′ end | 91 | no | TCTTCAATCTCTTGCACTCAAAGC | (15) | 98 | 24 | [symbol] 15 |
| | | | | TCCCCCAAATTCTAAGCAGAGT | (36) | | 22 | 805 |

[symbol]

indicates data missing or illegible when filed

mRNA transcript abundance was quantified during storage of blood stains using a qPCR reaction strategy in which the abundance of transcript fragments mapping to the 5′ end of the transcript and the 3′ end of the transcript were quantified in separate qPCR reactions in triplicate for each storage time point. This methodology (the 5′-3′ assay) allows for ΔCq to be computed by subtracting the Cq value of the 3′ amplicon from that produced for the 5′ amplicon. A qPCR assay was developed and the changes in the abundance of the 5′ and 3′ ends of several transcripts (i.e. ΔCq) was used to produce a standard curve comparing ΔCq values (i.e. Cq of the 5′ amplicon minus the Cq of the 3′ amplicon) versus storage time.

In one non-limiting example of the presently disclosed and/or claimed inventive concept(s), the accumulation of qPCR amplicons was quantified in ten microliter reactions using SYBR green intercalating dye. Reactions contained 5 μL PowerUp SYBR Green Master Mix (Thermo Fisher, Waltham, Mass.), 1 μL of 10× primer pair (8 μM or 3 μM, depending upon the particular primer), 3 μL H2O and 1 μL of 1:5 diluted cDNA. One non-limiting example of a cycling program used for qPCR was 50° C. for 2 min and 95° C. for 2 min followed by 40 cycles of 95° C. for 15 sec and 60° C. for 1 min. Cq values were determined manually using a threshold of 0.2, a standard baseline range (3-15), or a modified baseline range determined considering the kinetics of accumulating fluorescent signal. Negative qPCR controls consisted of reactions lacking added cDNA template. Cq results from negative controls ranged from “undetermined” to 33-36, which is at or near the limit of detection for the instrumentation used.

Data Analysis: ΔCq values for each timepoint during storage were plotted using a spreadsheet program commonly known in the art to produce degradation curves. ΔCq values for the 5′ and 3′ amplicons were produced in the following way: Cq results from triplicate qPCR reactions were averaged for each blood stain produced from each sample donor. ΔCq values were produced for each blood stain from each donor by subtracting the average Cq value for the 3′ amplicon from the average Cq value from the 5′ amplicon. The ΔCq values for each blood stain from each donor were then averaged together and plotted with error bars representing the variability in average ΔCq values from the different blood stain donors.

Statistical analysis of ΔCq data involved analysis of variance (ANOVA) methods to assess a possible correlation of ΔCq with storage time for each transcript. Means were further compared using post-hoc comparisons with a Tukey-Kramer adjustment. The ultimate goal of the statistical evaluation of RNA degradation data was to determine the window of possible error that would accompany any estimate of sample age. Statistical analysis was performed using SPSS (IBM Analytics, NY, NY) or SAS (Cary, N.C.) software programs. In one non-limiting embodiment, the order of transcript abundance in newly prepared bloodstains is (from highest to lowest): B2M (Cq=15-16)>S100A12 (Cq=19-20)>CLC (Cq=21-22)>LGALS2 (Cq=24-26). 5′ and 3′ amplicons produced from the LGALS2, CLC, and S100A12 transcripts were located within the coding region of the mRNAs, whereas the 5′ amplicon from the B2M transcript was from the coding region, but the 3′ amplicon was located within the 3′ untranslated region (FIG. 1).

As previously discussed, ΔCq results reflecting the degradation of the four transcripts are plotted in FIG. 2 and demonstrate the relationship between ΔCq and storage of stains at room temperature in a dry and dark environment for periods of up to one year. Of the four markers, the S100A12 transcript exhibits fairly linear degradation kinetics over the entire 52-week storage term with an R2 value of 0.898 (FIG. 2).

To estimate precision limits, statistical analysis of the ΔCq data from each of the degradation curves was performed using one-way ANOVA (with Tukey-Kramer post-hoc adjustments). Results of that analysis suggest that sample age estimates are accurate to within 2-4 weeks for samples stored less than 6 months and to within 4-6 weeks for samples stored between 6 months and one year.

The four transcript markers and their respective degradation curves were used to estimate the ages of bloodstains whose exact age was known (however, unknown to the analyst performing the experiment). For these experiments extra, archived stains prepared for the time course experiments shown in FIG. 2 were used. ΔCq values obtained from the unknowns were compared against the different degradation curves to produce an estimate of the sample age. For these estimates, a linear regression was used. Results of that exercise produced the results shown hereinbelow in Table 2 which shows estimates of the age of bloodstains obtained from the 5′-3′ qPCR assay and compares these estimates with the actual age of stains.

TABLE 2
Bloodstain Sample Age Estimation Utilizing LGAL, CLC, S100A12, and B2M Transcripts

| | | LGAL Estimated Age | CLC Estimated Age | S100A12 Estimated Age | B2M Estimated Age |
|---|---|---|---|---|---|
| Sample | Actual Age | R2 = 0.8467 | R2 = 0.741 | R2 = 0.8988 | N/A |
| 1 | 5 | 4 | 1 | 8 | 3 |
| 2 | 20 | 18 | 12 | 19 | >10 |
| 3 | 20 | 19 | 12 | 19 | >10 |
| 4 | 30 | 34 | 33 | 35 | >10 |
| 5 | 40 | 38 | 34 | 38 | >10 |
| 6 | 40 | 39 | 42 | 44 | >10 |
| 7 | 40 | 35 | 31 | 41 | >10 |
| 8 | 49 | 43 | 38 | 46 | >10 |

If the ΔCq-qPCR data is fitted to a standard curve based upon a regression employing either a quadratic or cubic equation, R² values are higher (i.e. 0.95 or 0.97 respectively). Equations reflecting a fit of the ΔCq data to linear, quadratic, and cubic formulae are shown in Table 3 hereinbelow along with R2 values for goodness of fit.

TABLE 3
ΔCq Data for LGALS2, CLC, S100A12, and B2M Transcripts Fit to Linear, Quadratic, and Cubic Formulae

| | Linear (0-52 weeks) | Quadratic (0-52 weeks) | Cubic fitting (0-52 weeks) |
|---|---|---|---|
| ΔCq-LGALS2 | Y = 0.0399*X + 0.8201 | Y = 0.47 + 0.09X − 0.00103X² | Y = 0.38 + 0.12X − 0.00253X² + 0.0000201X² |
| | R2 = 0.8467 | R² = 0.945 | R² = 0.953 |
| ΔCq-CLC | Y = 0.0414*X + 0.6044 | Y = 0.11 + 0.11X − 0.00147X² | Y = 0.04 + 0.16X − 0.0039X² + 0.0000326X² |
| | R2 = 0.741 | R² = 0.912 | R² = 0.930 |
| ΔCq-S100A12 | Y = 0.0337*X + 0.1164 | Y = 0.1 + 0.06X − 0.000649X² | Y = 0.21 + 0.1X − 0.00238X² + 0.0000232X² |
| | R2 = 0.8988 | R² = 0.942 | R² = 0.959 |
| ΔCq-82M | | Y = 0.14 + 0.07X − 0.00104X² | Y = 0.11 + 0.15X − 0.00524X² + 0.0000565X² |
| | | R² = 0.628 | R² = 0.767 |

The best fit of year-long storage data is observed using a cubic equation (Table 3). It should be emphasized however that determining the best fit of the data based upon the R2 values included ΔCq data for the entire year of sample storage.

Thus, in accordance with the presently disclosed and claimed inventive concept(s), there have been provided methods and kits related to estimation of the age of a particular biological fluid sample (either liquid or dried). As described herein, the presently disclosed and claimed inventive concept(s) relate to embodiments of a method for estimating the age of a particular biological fluid sample via quantifying the degradation of the 5′ and/or 3′ ends of a single ribonucleic acid transcript, including, without limitation, mRNA transcripts, as well as kits related thereto. Such presently disclosed and/or claimed inventive concept(s) fully satisfy the objectives and advantages set forth hereinabove. Although the presently disclosed and claimed inventive concept(s) has been described in conjunction with the specific drawings, experimentation, results and language set forth hereinabove, it is evident that many alternatives, modifications, and variations will be apparent to those skilled in the art. Accordingly, it is intended to embrace all such alternatives, modifications and variations that fall within the spirit and broad scope of the presently disclosed and claimed inventive concept(s).

What is claimed is:

1. A method for estimating the age of a biological fluid sample by detecting and quantifying the degradation of a ribonucleic acid transcript, the method comprising the steps of:
designing a first pair of primers to amplify a portion of a 5′ end of a ribonucleic acid transcript present in a biological fluid sample;
designing a second pair of primers to amplify a portion of a 3′ end of the ribonucleic acid transcript present in the biological fluid sample;
amplifying the portion of the 5′ end of the ribonucleic acid transcript present in the biological fluid sample utilizing the first pair of primers and the portion of the 3′ end of the ribonucleic acid transcript in the biological sample utilizing the second pair of primers;
quantifying the degradation of the ribonucleic acid transcript over a period of time to determine a point in time in which the ribonucleic acid transcript is present or not present in the biological fluid sample; and
estimating an age of the biological sample based on the present or non-presence of the ribonucleic acid transcript in the biological fluid sample.

2. The method of claim 1, wherein the portion of the 5′ end of the ribonucleic acid transcript is about 100 base pairs.

3. The method of claim 1, wherein the portion of the 3′ end of the ribonucleic acid transcript is about 100 base pairs.

4. The method of claim 1, wherein the ribonucleic acid transcript comprises a messenger ribonucleic acid transcript.

5. The method of claim 4, wherein the messenger ribonucleic acid transcript is selected from the group consisting of for galectin 2 (LGALS2), Charcot-Leyden crystal galectin (CLC), S100 calcium binding protein A12 (S100A12), beta-2-microglobulin (B2M), and combinations thereof.

6. The method of claim 1, wherein the biological fluid sample is selected from the group consisting of whole blood, blood plasma, blood serum, saliva, sputum, cerebrospinal fluid (CSF), vaginal fluid, intestinal fluid, intraperotineal fluid, cystic fluid, sweat, interstitial fluid, tears, mucus, urine, bladder wash, semen, and combinations thereof.

7. The method of claim 6, wherein the biological fluid sample is whole blood.

8. The method of claim 1, wherein the biological fluid sample is a dried sample.

9. The method of claim 1, wherein the biological fluid sample is a liquid sample.

10. The method of claim 1, wherein the portion of the 5′ end of the ribonucleic acid transcript and the portion of the 3′ end of the ribonucleic acid transcript are amplified via RT-PCR or qPCR.

11. The method of claim 1, wherein the period of time is in a range of from about 0 days to about 365 days.

12. The method of claim 1, wherein the degradation of the ribonucleic acid transcript is quantified by calculating a ΔCq value for the ribonucleic acid transcript.

13. The method of claim 12, wherein the ΔCq value is calculated by subtracting an amount of the 5′ end of the ribonucleic acid transcript from the amount of the 3′ end of the ribonucleic acid transcript.

14. A kit for estimating the age of a biological fluid sample by detecting and quantifying the degradation of a ribonucleic acid transcript, comprising:
a first pair of primers to amplify a portion of a 5′ end of a ribonucleic acid transcript present in a biological fluid sample;
a second pair of primers to amplify a portion of a 3′ end of the ribonucleic acid transcript present in a biological fluid sample; and
a printed correlation index that details degradation rates of common ribonucleic acid transcripts over a period of time.

15. The kit of claim 14, wherein the portion of the 5′ end of the ribonucleic acid transcript is about 100 base pairs.

16. The kit of claim 14, wherein the portion of the 3′ end of the ribonucleic acid transcript is about 100 base pairs.

17. The kit of claim 14, wherein the ribonucleic acid transcript comprises a messenger ribonucleic acid transcript.

18. The kit of claim 14, wherein the common ribonucleic acid transcripts are selected from the group consisting of galectin 2 (LGALS2), Charcot-Leyden crystal galectin (CLC), S100 calcium binding protein A12 (S100A12, beta-2-microglobulin (B2M), and combinations thereof.

19. The kit of claim 14, wherein the biological fluid sample is selected from the group consisting of whole blood, blood plasma, blood serum, saliva, sputum, cerebrospinal fluid (CSF), vaginal fluid, intestinal fluid, intraperotineal fluid, cystic fluid, sweat, interstitial fluid, tears, mucus, urine, bladder wash, semen, and combinations thereof.

20. The kit of claim 14, wherein the biological fluid sample is a dried sample.

21. The kit of claim 14, wherein the biological fluid sample is a liquid sample.


SEQUENCE LISTING (16 sequences)

SEQ ID NO: 1 - DNA, 21 bp, Homo sapiens
cgggggaacttgaggttaaga

SEQ ID NO: 2 - DNA, 22 bp, Homo sapiens
ttacaaagccatcagtgccatc

SEQ ID NO: 3 - DNA, 20 bp, Homo sapiens
atgggcacgagctgactttt

SEQ ID NO: 4 - DNA, 24 bp, Homo sapiens
cttgaaagaggacatgttgaaccc

SEQ ID NO: 5 - DNA, 21 bp, Homo sapiens
ggagacaacaatgtccctgct

SEQ ID NO: 6 - DNA, 21 bp, Homo sapiens
agtggtcgccctttgattgtc

SEQ ID NO: 7 - DNA, 21 bp, Homo sapiens
atggtgcaagtgtggagagat

SEQ ID NO: 8 - DNA, 21 bp, Homo sapiens
agggattccttggcaacatga

SEQ ID NO: 9 - DNA, 21 bp, Homo sapiens
ggggttaacattaggctggga

SEQ ID NO: 10 - DNA, 20 bp, Homo sapiens
tgtcaaaatgccccttccga

SEQ ID NO: 11 - DNA, 21 bp, Homo sapiens
tccaaggcctggatgctaatc

SEQ ID NO: 12 - DNA, 20 bp, Homo sapiens
tgtggtaatgggcagccttc

SEQ ID NO: 13 - DNA, 20 bp, Homo sapiens
tggaggctatccagcgtact

SEQ ID NO: 14 - DNA, 22 bp, Homo sapiens
cccagacacatagcaattcagg

SEQ ID NO: 15 - DNA, 24 bp, Homo sapiens
tcttcaatctcttgcactcaaagc

SEQ ID NO: 16 - DNA, 22 bp, Homo sapiens
tcccccaaattctaagcagagt